(19) United States
(12) Patent Application Publication
(10) Pub. No.: US 2009/0053696 A1
(43) Pub. Date: Feb. 26, 2009

(54) Biomarker For Heart Failure

(76) Inventors: Walter J. Koch, Philadelphia, PA (US); Guido Iaccarino, Naples (IT)

Correspondence Address: NIXON & VANDERHYE, PC, 901 NORTH GLEBE ROAD, 11TH FLOOR, ARLINGTON, VA, 22203, US

(21) Appl. No.: 11/667,013

(22) PCT Filed: Nov. 4, 2005
(86) PCT No.: PCT/US05/40231

§ 371 Date: May 4, 2007

Related U.S. Application Data
(60) Provisional application No. 60/625,719, filed on Nov. 8, 2004.

(51) Int. Cl. C12Q 1/68; G01N 33/53

(52) U.S. Cl. 435/6; 435/7.92

(57) ABSTRACT

The present invention relates, in general, to heart failure, and, in particular, to a method of evaluating heart failure patients by monitoring β-adrenergic receptor kinase (βARK1) levels in lymphocytes from such patients.

This application claims priority from Provisional Application. No. 60/625,719, filed Nov. 8, 2004, the content of which is incorporated herein by reference.

### TECHNICAL FIELD

The present invention relates, in general, to heart failure, and, in particular, to a method of evaluating heart failure patients by monitoring β-adrenergic receptor kinase (βARK1 or GRK2) levels in lymphocytes from such patients.

### BACKGROUND

β-adrenergic receptors (βARs) directly mediate the sympathetic nervous system control of cardiac inotropy and chronotropy. The adult cardiac myocyte expresses primarily β₁- and β₂-ARs, with the β₁-AR being the most abundant subtype (>75%) (Brodde, Basic Res Cardiol. 91:35-40 (1996)). After agonist binding, both subtypes couple primarily to the G protein, Gs, leading to the activation of adenylyl cyclase and enhanced production of the second messenger cAMP in the cardiac myocyte (Stiles et al, Cardiac adrenergic receptors. Annu Rev Med. 35:149-64 (1984)). In chronic human heart failure (HF), deterioration of ventricular function is associated with alterations of cardiac βAR signaling, including both a reduction of β₁-AR density and the functional uncoupling of remaining βARs (Rockman et al, Nature 415:206-12 (2002)). This latter phenomenon is known as desensitization and is triggered by the phosphorylation of agonist-occupied βARs by G protein coupled receptor (GPCR) kinases (GRKs) (Rockman et al, Nature 415:206-12 (2002)). Both β₁- and β₂-ARs can be phosphorylated by GRKs and in the heart, the prominent GRK appears to be GRK2, also known as the βAR kinase (βARK1) (Lefkowitz, Cell. 74:409-12 (1993)).

βARK1 (or GRK2) is a cytosolic enzyme that localizes to the membrane through binding to the G_{βγ} subunits of activated heterotrimeric G proteins (Rockman et al, Nature 415:206-12 (2002), Lefkowitz, Cell. 74:409-12 (1993), Pierce et al, Nat Rev Mol Cell Biol. 3:639-50 (2002)). It plays a role in the control of cardiac βAR signaling and function as demonstrated in transgenic mice with myocardial overexpression of the kinase (Koch et al, Science 268:1350-3 (1995)). In these mice, cAMP production and cardiac contractility in response to βAR stimulation was significantly reduced when βARK1 was increased 3-4 fold (Koch et al, Science 268:1350-3 (1995)). Moreover, studies in mice where βARK1 activity or expression were reduced in the heart showed an increase in βAR signaling and cardiac function (Koch et al, Science 268:1350-3 (1995), Rockman et al, J Biol. Chem. 273:18180-4 (1998)). These studies were the first to demonstrate, in vivo, the critical dependence of βARK1 levels on cardiac βAR signaling. Myocardial levels of βARK1 appear to be actively regulated, since in human HF as well as in animal models, there is a characteristic elevation of myocardial expression and activity of βARK1 (Ungerer et al, Circulation 87:454-63 (1993), Ungerer et al, Circ. Res. 74:206-13 (1994), Maurice et al, Am. J. Physiol. 276:H1853-60 (1999), Anderson et al, Hypertension. 33:402-7 (1999), Rockman et al, Proc. Natl. Acad. Sci. USA. 95:7000-5 (1998), Ping et al, Am J. Physiol. 273:H707-17 (1997), Harris et al, Basic Res Cardiol. 96:364-8 (2001)). This increase in βARK1 (2-3 fold) appears responsible for the enhanced βAR desensitization seen in compromised myocardium (Rockman et al, Proc. Natl. Acad. Sci. U S A. 95:7000-5 (1998), Ping et al, Am J Physiol. 273:H707-17 (1997), Harris et al, Basic Res Cardiol. 96:364-8 (2001), White et al, Proc. Natl. Acad. Sci. U S A. 97:5428-33 (2000)). βARK1 appears to be the primary βAR regulatory molecule altered in human HF as P-arrestins and GRK3 are not altered in failing human hearts (Ungerer et al, Circulation 87:454-63 (1993), Ungerer et al, Circ. Res. 74:206-13 (1994)). GRK5, another major GRK in myocardium, has not been studied in human HF although it has been shown to be up-regulated in some animal models (Ping et al, Am J Physiol. 273:H707-17 (1997), Vinge et al, Am. J. Physiol. 281:H2490-9 (2001)).

The relevance of the molecular abnormalities of βAR signaling to the pathogenesis of human HF, and perhaps more importantly to HF outcome are not completely understood. An important aspect of βAR signaling is that properties of the system in circulating white blood cells appear to mirror those observed in solid tissues. This was first observed in the heart in 1986 (Brodde et al, Science 231:1584-5 (1986)) and many other reports have also used the lymphocyte system to study βAR signaling and to make extrapolations to the cardiac βAR system (Bristow et al, Clin. Investig. 70:S105-13 (1992), Jones et al, J. Cardiovasc. Pharmacol. 8:562-6 (1986), Sun et al, Crit. Care Med. 24:1654-9 (1996), Dzimiri et al, Clin Exp Pharmacol Physiol. 23:498-502 (1996)).

Much data has recently accumulated in experimental models suggesting that the increased βARK1 expression and activity in failing myocardium can contribute to the pathogenesis of HF (Rockman et al, Nature 415:206-12 (2002)). The present invention results, at least in part, from studies designed to investigate the value of cardiac βAR signaling and βARK1 activity in the evolution and severity of human HF. These studies have demonstrated that blood and cardiac (right atrium) βARK1 levels correlate in a direct fashion. The invention thus provides a method of assessing HF severity by monitoring lymphocyte βARK1 content and activity.

### SUMMARY OF THE INVENTION

The present invention relates to a method of assessing the status of HF patients by monitoring βARK1 levels in lymphocytes of such patients. Elevated βARK1 levels in lymphocytes correlate with elevated cardiac βARK1 levels and are associated with an unfavorable prognosis.

Objects and advantages of the present invention will be clear from the description that follows.

### BRIEF DESCRIPTION OF THE DRAWINGS

FIGS. 1A-1C. (FIG. 1A) Graph showing the direct correlation between soluble GRK activity measured by the in vitro phosphorylation of rhodopsin and βARK1 expression detected by protein immunoblotting. (FIG. 1B) Graph showing an inverse correlation between soluble GRK activity and isoproterenol (ISO) stimulation of adenylyl cyclase activity in cardiac membranes from LV biopsies from explanted failing human hearts. Adenylyl cyclase activity is plotted by the % ISO response over basal stimulation (n=24, p<0.05). (FIG. 1C) Using a similar approach in the same samples, a direct correlation was observed between βAR density and βAR signaling (ISO-stimulated adenylyl cyclase activity over basal stimulation, n=24, p<0.0001).

FIGS. 2A and 2B. (FIG. 2A) Graph showing the direct correlation between βARK1 expression in the heart (right atrial biopsies) and in the lymphocytes of HF patients. βARK1 expression was assessed by protein immunoblotting and the data is expressed as arbitrary densitometry units. (FIG. 2B) Representative autoradiograph from a protein immunoblot showing βARK1 expression in lymphocyte extracts and in extracts from right atrial appendages from the same sets of human HF patients (#37 and #53) with different degrees of ventricular dysfunction.

FIGS. 3A-3C. (FIG. 3A) Graph showing the inverse relationship between soluble GRK activity and cardiac function (% LV ejection fraction (EF)) assessed in HF patients (n=55, p<0.02). (FIG. 3B) Using a cut off of 45% LVEF, the 55 HF patients were divided into two groups. Those showing reduced cardiac function also had higher lymphocyte soluble GRK activity. *, p<0.05 (Unpaired Student's t-test). (FIG. 3C) When patients were stratified according to their NYHA HF class, there was a significant and progressive increase in lymphocyte soluble GRK activity.

FIGS. 4A and 4B. Paired samples from failing human LVs were obtained at the time of LVAD implantation and subsequent cardiac transplantation and βARK1 protein (FIG. 4A) and mRNA (FIG. 4B) was measured (n=12). (FIG. 4A) Results (mean ±SEM) of βARK1 immunoblotting in pre-(core) and post-LVAD (LV) samples with a representative Western blot shown. (+) control is purified βARK1. *, P<0.005 vs. pre-LVAD values. (FIG. 4B) Real-time quantitative RT-PCR of same samples (n=12) using SYBR® green fluorescence methodology. *, P<0.05 vs. pre-LVAD values (Paired Student's t-test).

FIGS. 5A and 5B. (FIG. 5A) Cardiac soluble GRK activity (mean ±SEM) found in cardiac samples pre- and post-LVAD (n=4 pairs). Soluble cardiac lysates were purified as described and incubated with [³²P-ATP] and purified rod outer segment membranes enriched with the GPCR rhodopsin (Rho) (Choi et al, J. Biol. Chem. 272:17223-17229 (1997), Iaccarino et al, Circulation 98:1783-1789 (1998)). Shown in the inset is an autoradiography of phosphor-incorporation into Rho after gel electrophoresis. *, P<0.05 vs. pre-LVAD values (t-test). (FIG. 5B) Membrane AC activity in cardiac lysates from paired pre- and post-LVAD LV samples (n=4). Data shown is the mean ±SEM of the % ISO-stimulation over basal activity showing a significant increase in βAR responsiveness. P<0.05 vs. pre-LVAD.

FIGS. 6A and 6B. (FIG. 6A) Lymphocyte βARK1 protein levels in blood sample obtained from two patients prior to LVAD implantation (Pre) and after explantation (Post). The mean data of the above Western is shown in the histogram. Purified βARK1 is the (+) control. (FIG. 6B) Cardiac GRK5 protein levels in paired samples pre-(core) and post-LVAD (LV). Data is mean ±SEM of n=15 pairs of samples in relative densitometry units of scanned Western blots. A representative immunoblot is shown in the inset with purified GRK5 as the (+) control.

### DETAILED DESCRIPTION OF THE INVENTION

The present invention relates to a method of assessing patients with HF by measuring lymphocyte βARK1 levels. The present invention results from studies demonstrating that blood and cardiac βARK1 levels and GRK activity correlate in a direct fashion. Thus, lymphocyte βARK1 content can serve as an easily accessible means of monitoring cardiac βARK1 levels and providing an indication of myocardial βAR signaling and HF severity. βARK1 levels and/or activity can be monitored to assess progression of therapy in HF, an elevated level of βARK1 being associated with the loss of βAR responsiveness and an unfavorable prognosis of a HF patient.

In accordance with the present invention, lymphocytes can be collected from patients and assayed for βARK1 protein levels, GRK activity and/or βARK1 mRNA content. More specifically, patient blood can be collected and anticoagulated using, for example EDTA. Lymphocytes can be isolated by Ficoll gradient (Chuang et al, J. Biol. Chem. 267:6886-6892 (1992)), or other convenient means. The lymphocytes can then be further processed or stored frozen (e.g., at −80° C.).

βARK1 protein levels can be determined using any of a variety of methods. For example, lymphocytes can be processed and lysed using detergent-containing buffers (Iaccarino et al, Circulation 98:1783-1789 (1998)) and βARK1 protein levels in cytosolic extracts can be detected by an ELISA technique (Oppermann et al, J. Biol. Chem. 274:8875-8885 (1999)) or Western blotting using βARK1 specific antibodies (monoclonal or polyclonal). Examples of suitable antibodies include the polyclonal antibodies (C-20) from Santa Cruz Biotechnology (catalogue number SC-561) and monoclonal antibodies raised against, for example, an epitope within the carboxyl terminus of βARK1 (Oppermann et al, Proc. Natl. Acad. Sci. USA 93:7649 (1996)). Such antibodies are commercially available, for example, through Upstate (e.g., clone C5/1, catalogue number 05-465). Quantitation of immunoreactive βARK1 can be effected by scanning the resulting autoradiographic film using, for example, ImageQuant software. Alternatively, visualization of βARK1 can be effected using standard enhanced chemiluminescence (Iaccarino et al, Circulation 98:1783-1789 (1998)), kits for which are commercially available. Other approaches to determining βARK1 protein levels include an ELISA method and immunofluorescence (Oppermann et al, J. Biol. Chem. 274:8875-8885 (1999)). While reference is made above to the use of lymphocytes, βARK1 levels can potentially be measured using serum.

In addition to βARK1 protein levels, cytosolic GRK activity can also be assayed in the cell extracts (Iaccarino et al, Circulation 98:1783-1789 (1998)). While any convenient means can be used, preferred are assays based on light-dependent phosphorylation of rhodopsin-enriched rod outer segment membrane using [γ-³²P]-ATP (Iaccarino et al, Circulation 98:1783-1789 (1998), Iaccarino et al, Hypertension 33:396-401 (1999), Iaccarino et al, J. Amer. Coll. Cardiol. 38:55-60 (2001), Choi et al, J. Biol. Chem. 272:17223-17229 (1997)). Soluble GRK activity represents primarily βARK1 activity. (See also De Blasi et al, J. Clin. Invest. 95:203-210 (1995).) In addition to rhodopsin, GRK2 activity can be assayed using suitable peptide substrates (Pitcher et al, J. Biol. Chem. 271:24907-24913 (1996)).

As indicated above, the present method can also be based on the determination βARK1 mRNA levels in lymphocytes. βARK1 mRNA can be determined using any of a variety of approaches, including Northern blot analysis (see, for example, De Blasi et al, J. Clin. Invest. 95:203-210 (1995)) or real time quantitative RT-PCR using SYBR green fluorescence methodology (Most et al, J. Clin. Invest. in press (December 2004)).

It will be appreciated form a reading of the foregoing that the present approach can be used at the stage of initial patient screening, where βARK1 protein, mRNA and/or activity levels present in a patient's lymphocytes are compared to control (non-diseased) levels. Available data indicate that normal (control) levels of βARK1 protein are approximately 100 ng/ml whole blood. Increases of about 50% or more over control levels can be considered “high”. In practice, βARK1 levels can be correlated with baseline cardiac function of the patient. The instant method can also be used to track the patient's status (e.g., following therapeutic intervention) by comparing the lymphocyte levels of βARK1 protein, mRNA and/or activity at different points in time after initiation of various regimens (e.g., drug regimens). The invention thus provides a method of monitoring the effects of therapy (e.g., the use ACE inhibitors, AT1 antagonists, and β-blockers) and procedures (including βAR blockade) on βAR signaling. When the opportunity arises (such as during cardiac surgery) myocardial tissue samples can be taken to ensure correlation between blood and tissue βARK1 levels.

The data presented in the Examples that follow demonstrate a critical relevance of βARK1 in the setting of βAR dysfunction in the human heart. Specifically, the data indicate that measuring βARK1 in blood samples can be used to monitor relative expression levels of this GRK in myocardium. Moreover, lymphocyte βARK1 content and activity in human HF patients appear to track with disease severity and thus are of prognostic use.

Certain aspects of the invention can be described in greater detail in the non-limiting Examples that follows.

### EXAMPLE 1

### Experimental Details

### Study Population

Three groups of patients were studied. The first group consisted of 24 patients undergoing cardiac transplantation due to severe functional deterioration and presented with the clinical characteristics indicated in Table 1 (Group 1). A second group included 55 patients that were admitted into the intensive care unit with various degree of cardiac dysfunction (Group 3). Among this group, 10 patients underwent elective cardiac surgery (Table 1, Group 2). All procedures were performed in compliance to Institutional guidelines.

TABLE 1
Clinical Characteristics of Patients Analyzed in this Study.

| | Group 1 | Group 2 | Group 3 |
|---|---|---|---|
| n-size | 24 | 10 | 55 |
| NYHA Class | 3-4 | 1-3 | 1-4 |
| Age (years) | 60 ± 2 | 71 ± 2 | 65 ± 2 |
| Sex (% M/% F) | 70/30 | 86/14 | 65/35 |
| Ishemic/Dilated Cardiomyopathy | 50/50 | n.a | n.a |
| (%) | | | |
| Diabetes (%) | 17 | 40 | 25 |
| Hypertension (%) | 33 | 20 | 23 |
| Dyslipidemia | 20 | 40 | 17 |
| Beta blockade (%) | 8 | 20 | 22 |
| ACE inhibition (%) | 50 | 50 | 58 |
| AR Blockade (%) | 58 | 0 | 8 |
| Diuretics (%) | 42 | 40 | 19 |
| Ca Antagonists (%) | 25 | 90 | 31 |
| Nitrates (%) | 42 | 70 | 69 |
| Digoxin (%) | 25 | 20 | 50 |

### Myocardial Samples

Following blood-buffered cardioplegia, transmural left ventricular (LV) tissue (≈2 grams wet weight) specimens from failing hearts was obtained during cardiac transplantation from 24 patients with HF due to ischemic or dilated cardiomyopathy. Right atrial appendages (≈200 mg wet weight) were also obtained from Group 2 patients undergoing cardiac surgery (aortocoronary bypass grafting or valvular replacement). Immediately after removal, all specimens were placed in ice-cold saline, rinsed, frozen in liquid nitrogen and stored at −80° C.

### Peripheral Lymphocyte Samples

Blood was collected and anticoagulated with EDTA. In Group 2 patients, blood was collected on the day before surgery. Lymphocytes were isolated by Ficoll gradient using HISTOPAQUE-1077 (Sigma), frozen and stored at −80° C. until the day of the assay (Bristow et al, Clin. Investig. 70:S105-13 (1992), Sun et al, Crit. Care Med. 24:1654-9 (1996)).

### βAR Density and Membrane Adenylyl Cyclase Activity Assays

Crude myocardial membranes were prepared from myocardial biopsies or lymphocytes as previously described (Iaccarino et al, Circulation. 98:1783-9 (1998), Iaccarino et al, Hypertension. 33:396-401 (1999)). βAR density was determined by radioligand binding with the non-selective βAR ligand [¹²⁵I]-CYP and membrane adenylyl cyclase activity and cAMP, under basal conditions or in the presence of either 100 μmol/L isoproterenol (ISO) or 10 mmol/L NaF and cAMP, was quantified using standard methods (Iaccarino et al, Circulation. 98:1783-9 (1998), Iaccarino et al, Hypertension. 33:396-401 (1999)).

### Protein Immunoblotting

Immunodetection of myocardial levels of βARK1 were performed using detergent-solubilized cardiac extracts after immunoprecipitation (IP) as previously described (Iaccarino et al, Circulation. 98:1783-9 (1998), Iaccarino et al, Hypertension. 33:396-401 (1999)). IP's were done using a monoclonal anti-GRK2/GRK3 antibody (C5/1, Upstate Biotechnology) followed by Western blotting with a specific βARK1 (GRK2) polyclonal antibody (C-20, (catalogue number SC-561)) Santa Cruz Biotechnology) (Iaccarino et al, Circulation 98:1783-9 (1998), Iaccarino et al, Hypertension 33:396-401 (1999), Iaccarino et al, J. Amer. Coll. Cardiol. 38:55-60 (2001)). Quantitation of immunoreactive βARK1 was done by scanning the autoradiography film and using ImageQuant software (Molecular Dynamics) (Iaccarino et al, J. Amer. Coll. Cardiol. 38:55-60 (2001)).

### GRK Activity Assays

Extracts were prepared through homogenization of cardiac tissue or lymphocytes in 2 mL of ice-cold detergent-free lysis buffer. Cytosolic fractions and membrane fractions were separated by centrifugation and soluble GRK activity was assessed in cytosolic fractions (100 to 150 μg of protein) by light-dependent phosphorylation of rhodopsin-enriched rod outer segment membranes using [γ-³²P]-ATP (Iaccarino et al, Circulation. 98:1783-9 (1998), Iaccarino et al, Hypertension. 33:396-401 (1999), Iaccarino et al, J. Amer. Coll. Cardiol. 38:55-60 (2001), Choi et al, J. Biol. Chem. 272:17223-17229 (1997)). Soluble GRK activity represents primarily βARK1 activity and changes in βARK1 expression correlate with altered βAR signaling (Choi et al, J. Biol. Chem. 272:17223-17229 (1997)).

### Statistical Analysis.

Statistical analysis was performed using the Systat 7.0 software for Windows. Values are given as the mean ±SEM. To compare groups, a Student's unpaired t test was used. Correlations between variables were studied using the analysis of the linear regression test. Correlation was considered significant when the p value for the F test was less then 0.05. The effect of βAR density and GRK activity on adenylyl cyclase activity was also calculated using these as coefficients in a forward stepwise multiple regression analysis.

### Results

### β-Adrenergic Signaling in Failing Human Myocardium

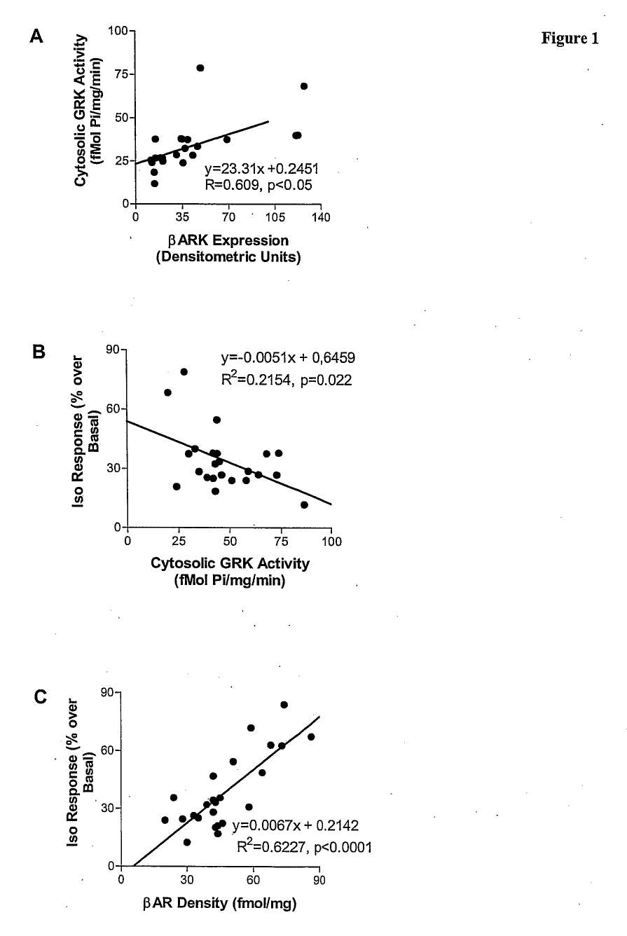

The clinical characteristics of the patients from whom heart tissue was obtained during transplantation (Group 1) are listed in Table 1. βARK1 expression and activity in cytosolic extracts from these failing heart samples was first assessed and it was found that there was a direct correlation between βARK1 protein and in vitro GRK activity (R=0.609, p<0.05; n=24) (FIG. 1A). Since experimental studies in animals has shown that levels of myocardial βARK1 can greatly influence βAR signaling in the heart (Koch et al, Science 268:1350-1353 (1995), Rockman et al, J. Biol. Chem. 273:18180-18184 (1998)), the relationship between βAR-mediated adenylyl cyclase activity in cardiac membranes and cytosolic βARK1 activity was evaluated. The relationship between βAR density and cAMP production was also assessed in the same failing heart biopsies. First, a significant inverse correlation was found between soluble GRK activity and βAR responsiveness. As FIG. 1B demonstrates, when GRK activity is greater, βAR signaling, as measured by ISO-stimulated adenylyl cyclase activity, is depressed. Further, as would be expected, there was a positive correlation found between ISO-mediated cAMP production and myocardial βAR density (FIG. 1C). Therefore, both βAR density and GRK activity significantly affect cAMP production, as indicated by the linear regression analysis, (F=31.861, p<0.001; βAR density: T: 6,285, p<0.001; GRK activity: T:−3,311, p<0.005)

To verify whether altered myocardial P-adrenergic signaling has any relationship to outcomes of human HF and whether βARK1 activity could be linked to the severity of the disease, soluble GRK activity in LV biopsies was measured and levels compared in patients with varying times between their initial diagnosis of HF to when the intervention of cardiac transplantation or implantation of a LV assist device was performed. The population used in this analysis consisted of 15 patients from Group 1 (Table 1) that had a rapid evolution of HF (<2 years). This time frame was arbitrarily chosen to avoid any confounding effects of adaptive mechanisms that could have occurred in patients with a longer history of disease. Within this group, 5 patients required intervention within 7 months after diagnosis and in these patients, cardiac soluble GRK activity (46±10 fmol Pi/mg protein/min) was significantly higher than found in myocardial extracts from the remaining 10 patients who had an intervention between 7 and 24 months after an initial HF diagnosis (30±2 fmol Pi/mg protein/min) (p<0.005, t test). Interestingly, in these same two groups there was no difference in myocardial βAR density (41±13 fmol/mg membrane protein versus 38±4 fmol/mg membrane protein) or adenylyl cyclase activity. Thus, although the small sample size was relatively small and cut-off conditions were selected post-hoc, these data suggest that cardiac βARK1 may be a more suitable predictor of disease severity and/or risk of progression than βAR density or coupling.

### β-Adrenergic Signaling in Peripheral Lymphocytes in HF

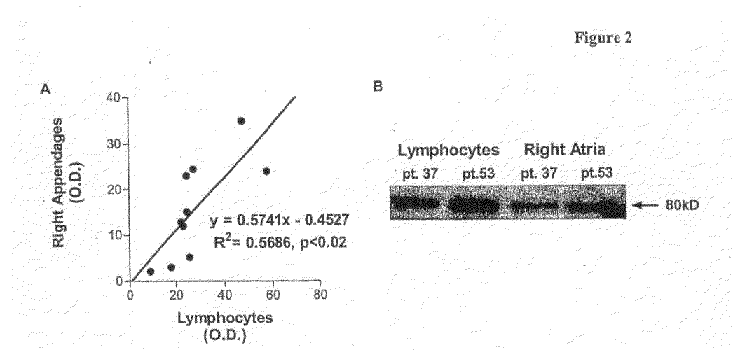

A hypothesis that was tested was whether the βAR system and in particular βARK1 in white blood cells could be used as a surrogate for what is seen in failing myocardium. In order to verify any correlation between cardiac and peripheral lymphocytes in terms of GRK activity, βARK1 expression in right atrial appendages from surgical biopsies and lymphocytes from patients in Group 2 patients (Table 1) was measured. These patients underwent surgery for coronary artery disease or valvular replacement and were generally in NYHA class 1-3 HF. As shown in FIG. 2A, a direct correlation was found between myocardial and lymphocyte βARK1 expression, indicating that lymphocyte levels of this GRK mirrors cardiac expression. Specifically, when βARK1 levels are elevated in the myocardium, this is also apparent in lymphocyte extracts. An example of this is shown in FIG. 2B in two HF patients with different disease severity.

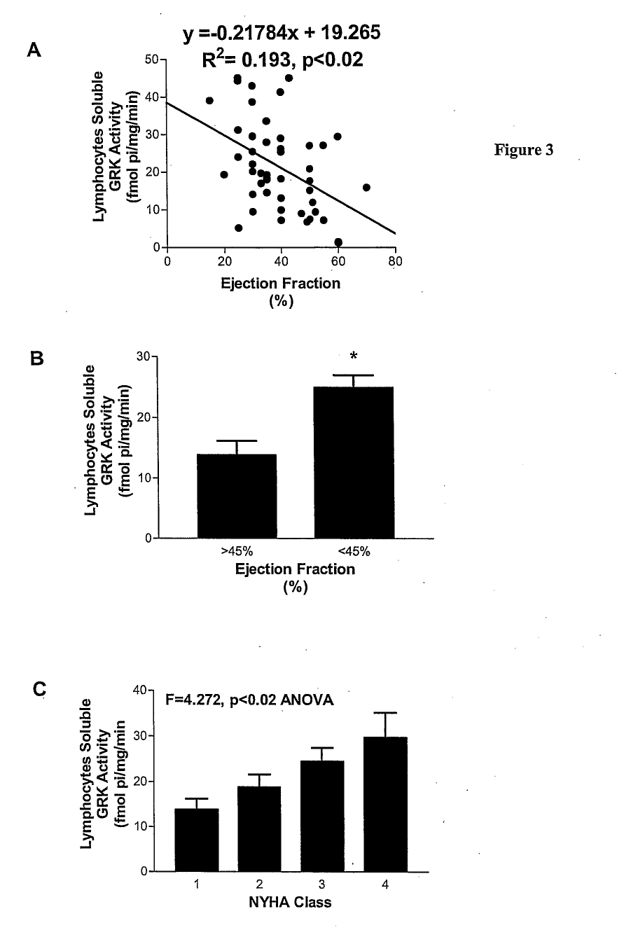

Based on this observation, lymphocyte βARK1 expression and GRK activity analysis was extended to a larger number of patients with different degrees of cardiac function, ranging from normal to significantly depressed (as assessed by echocardiography). The characteristics of these patients (Group 3) are listed in Table 1. Whether lymphocyte βARK1 content correlated with cardiac function was specifically addressed by plotting LV ejection fraction (LVEF, %) against soluble lymphocyte GRK activity. As shown in FIG. 3A, there is a statistically significant inverse correlation between LVEF and βARK1 activity in the blood of these 55 patients. This can be more clearly seen when this group is divided into two groups at a functional cut-off of 45% LVEF. Cytosol GRK activity is significantly higher in the white blood cells from patients with poorer LV function (FIG. 3B). Similarly, a stepwise increase in GRK activity with NYHA functional class was observed (FIG. 3C). Not taking into account all other variables in these patients such as exercise tolerance, specific drug treatments or other measurements of cardiac function, the use of LVEF appears to indicate that in patients with lower ventricular function, there are higher levels of cardiac βARK1 activity that can be measured in peripheral lymphocytes.

Summarizing, the study described above focuses on the role of the GRK, βARK1 (or GRK2) in human HF and provides three major novel observations: 1) the demonstration that increased cardiac βARK1 levels correlate with decreased βAR signaling in failing human hearts; 2) the direct demonstration that cardiac βARK1 levels and GRK activity can be monitored using peripheral lymphocytes; and 3) the suggestion that increased βARK1 may be associated with more rapidly progressive HF and adverse clinical outcome. These data indicate the usefulness in measuring blood levels of this GRK in HF patients during initial screening for this disease.

Several studies in animal models have provided a thorough analysis of the mechanisms by which βARK1 participates in the uncoupling of βAR signaling and the onset of HF (Rockman et al, Nature 415:206-12 (2002)). By contrast, only two studies have described increased levels of βARK1 in autopsy specimens from failing human hearts at the time of explantation (Ungerer et al, Circulation 87:454-63 (1993), Ungerer et al, Circ. Res. 74:206-13 (1994)). By assessing βARK1 and βAR signaling from similar LV biopsies taken at explantation, an inverse correlation was found between βARK1 and GRK activity and βAR signaling. This is important information to go along with existing knowledge that there is a direct correlation between myocardial βAR density and cardiac cAMP production in response to βAR stimulation. These data suggest a critical relevance of βARK1 in the setting of βAR dysfunction in the human heart. Key regulatory processes involved in βAR signaling are receptor desensitization and internalization, which are triggered by βAR phosphorylation by βARK1 or other GRKs (Rockman et al, Nature 415:206-12 (2002), Lefkowitz, Cell. 74:409-12 (1993), Pierce et al, Nat Rev Mol Cell Biol. 3:639-50 (2002)). It is possible that other mechanisms may also contribute to βAR dysfunction in HF such as the up-regulation of the α subunit of the cyclase inhibitory G protein Gi (Gαi), and altered expression of adenylyl cyclase isoforms (Bristow, J. Amer. Coll. Cardiol. 22:61A-71A (1993)). However, due to the fact that a significant inverse correlation was found between βAR responsiveness and GRK activity in the failing heart, it appears that βARK1 plays a critical role in human myocardial βAR regulation and function.

An additional significant finding of the study is the demonstration that there is a direct correlation between lymphocyte and cardiac (right atrial appendages) βARK1 expression and activity. Thus, measuring βARK1 in blood samples can be used to monitor relative expression levels of this GRK in myocardium. The possibility to use lymphocytes for monitoring drug- or disease-induced βAR changes in the heart, which is not easily accessible in humans, was first hypothesized by Brodde et al. (Science 231:1584-5 (1986)), and further realized by others (Feldman et al, J. Clin. Invest. 79:290-4 (1987)). The utility of monitoring components of βAR signaling in lymphocyte of HF patients has been proposed by several groups, however data is conflicting regarding the ultimate utility of measuring G proteins, βAR density and cAMP in lymphocytes (Brodde et al, Science 231:1584-5 (1986), Feldman et al, J. Clin. Invest. 79:290-4 (1987), Maisel et. al, Circulation 81:1198-204 (1990), Gros et al, J. Clin. Invest. 99:2087-93 (1997)). Concerning GRKs, evidence has been presented to support that increased lymphocyte βARK1 is a characteristic of certain cardiovascular pathologies including hypertension supporting the phenotypic intercurrence between cardiac and lymphocyte βAR systems (Feldman et al, J. Clin. Invest. 79:290-4 (1987), Maisel et. al, Circulation 81:1198-204 (1990), Gros et al, J. Clin. Invest. 99:2087-93 (1997)). The present study adds to this scenario by providing the novel finding that this system can be used to study the key βAR regulatory molecule βARK1 and its associated soluble GRK activity. Moreover, it appears that lymphocyte βARK1 content and activity in human HF patients may track with disease severity. Although the current data does not support the use of lymphocyte GRK monitoring as a predictor for individual patient outcomes, it does appear to be a potentially useful marker to explore in the initial screening and follow up of HF patients.

The mechanism responsible for similar alterations in the βAR system of lymphocytes and myocardium is uncertain. Recent data from Brodde and colleagues (Werner et al, Basic Res. Cardiol. 96:290-8 (2001)) show that βAR blockade in HF, a treatment that in animals reduces cardiac βARK1 and increases signaling (Iaccarino et al, Circulation. 98:1783-9 (1998)), can also increase functional and immune responses to catecholamines in lymphocytes (Werner et al, Basic Res. Cardiol. 96:290-8 (2001)). Signaling through the βAR system in these lymphocytes were increased regardless of the effects on cardiac function (Werner et al, Basic Res. Cardiol. 96:290-8 (2001)). These data support the concept that the GRK system in lymphocytes and the heart are regulated in a similar manner. It is known that chronic catecholamine exposure induces βAR signaling abnormalities such as βAR down-regulation and that HF is associated with increased circulating norepinephrine (Bristow, J. Amer. Coll. Cardiol. 22:61A-71A (1993), Hasking et al, Circulation 73:615-21 (1986)). Importantly, immune responses in HF patients can be modulated by the sympathetic nervous system and the underlying mechanism appears to involve β₂-ARs (Murray et al, Circulation 86:203-13 (1992)), which could occur through epinephrine stimulation, which is increased in HF patients (Kaye et al, Am. J. Physiol. 269:H182-8 (1995)). Since myocardial βARK1 is up-regulated in response to chronic adrenergic activation (Iaccarino et al, Circulation. 98:1783-9 (1998), Iaccarino et al, Hypertension. 33:396-401 (1999), Iaccarino et al, J. Amer. Coll. Cardiol. 38:55-60 (2001), Iaccarino et al, Hypertension 38:255-60 (2001)), one possibility is that the increased circulating catecholamines (i.e. norepinephrine and epinephrine), can trigger an increase in βARK1 expression both in the lymphocyte and in the heart through means of β₁- and β₂-AR stimulation. However, this hypothesis needs to be explored further in HF patients who have been treated with βAR antagonists to determine if blockade of chronic catecholamine activation of βARs in the heart and circulating white blood cells can indeed affect βARK1 expression. These further clinical studies will also be important to better define the relationship between lymphocyte βARK1 activity and myocardial adrenergic responsiveness. Interestingly, this has been shown to be the case in the hearts of mice chronically exposed to carvedilol and atenolol (Iaccarino et al, Circulation. 98:1783-9 (1998)), and in HF pigs treated with a β-blocker (Ping et al, J. Clin. Invest. 95:1271-80 (1995)). In vitro studies suggest that β-blockers reduce βARK1 expression through both reduction of βARK1 mRNA and protein (Iaccarino et al, Circulation. 98:1783-9 (1998)).

In animal models of HF, cardiac GRK activity up-regulation is frequently (Maurice et al, Am. J. Physiol. 276:H1853-60 (1999), Anderson et al, Hypertension. 33:402-7 (1999), Rockman et al, Proc. Natl. Acad. Sci. U S A. 95:7000-5 (1998), Ping et al, Am J Physiol. 273:H707-17 (1997), Harris et al, Basic Res Cardiol. 096:364-8 (2001), Iaccarino et al, J. Amer. Coll. Cardiol. 38:55-60 (2001), Akhter et al, Proc. Natl. Acad. Sci. USA. 94:12100-5 (1997), Asai et al, J. Clin. Invest. 104:551-8 (1999), Cho et al, J. Biol. Chem. 274:22251-6 (1999)), but not always (Dorn et al, Mol. Pharmacol. 57:278-87 (2000)), observed. This observation might suggest a differential role of this kinase in HF. In the present study, it was observed that decreased cardiac performance (i.e. LVEF) was not consistently associated with increased βARK1 levels. However, the data indicate that there may be a correlation between βARK1 and more negative outcomes in HF as in ischemic patients, higher cardiac GRK activity was associated with more rapidly progressive HF. These findings in humans parallel a recent study in transgenic mice, in which increased βARK1 expression and activity was associated with severe cardiomyopathy and early mortality (Iaccarino et al, J. Amer. Coll. Cardiol. 38:55-60 (2001)). A case for βARK1 representing a molecule to be monitored in human HF to predict disease severity is perhaps best illustrated in the findings that βARK1 was significantly and progressively higher with escalating NYHA HF class. This is similar to what has been shown for brain natriuretic peptide (BNP) (Lee et al, J. Card Failure 8:149-54 (2002)). Importantly, like BNP, βARK1 expression and activity in lymphocytes represents a novel and readily assessable biomarker for human HF. Overall, the data indicate measuring lymphocyte βARK1 levels is useful in the assessment of patients with HF. Studies involving larger populations can be used to clarify the predictive role for βARK1 in HF.

### EXAMPLE 2

A study has been carried out involving the use of hearts of patients that have undergone surgery for implantation of a LV mechanical assist device (LVAD). These patients typically undergo cardiac transplantation within a few months and thus, heart samples before and after unloading can be obtained. Importantly, LVAD use as a “bridge-to-transplant” has been shown to lead to recovery of failing myocardium, a process termed reverse remodeling (Zafeiridis et al, Circulation 98:656-662 (1998)). Since previous studies have shown normalization of cardiac structure and function as a characteristic of post-LVAD reverse remodeling that includes improved βAR responsiveness (Zafeiridis et al, Circulation 98:656-662 (1998)), it was posited that βARK1 could be involved in this process. Cardiac βARK1 mRNA, protein and GRK activity has been measured in pre- and post-LVAD human LV samples. By using paired samples, it is possible to examine βARK1 specifically in the same heart before and after LVAD support. Initial results clearly show that βARK1 is reduced in the failing heart after a period of unloading (FIG. 4).

As shown in the Western blot (FIG. 4A), although pre-LVAD βARK1 protein amounts are variable, there is significant reduction after LVAD-mediated unloading. The average length of time for LVAD use in these patients was 2 months. βARK1 mRNA was quantitated using real-time RT-PCR using SYBR® green fluorescence methodology and this also showed significant reduction of βARK1 expression after unloading in human HF (FIG. 4B). Both βARK1 mRNA and protein were reduced by approximately 50%.

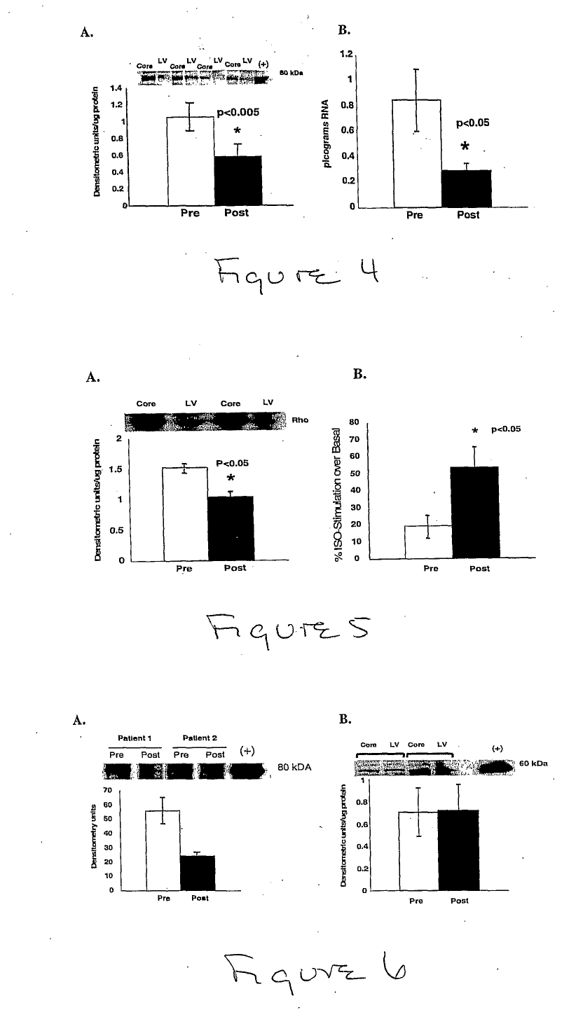

Cardiac GRK activity and βAR signaling were also examined in paired sets of human HF pre- and post-LVAD samples and preliminary results are shown in FIG. 5. Consistent with the mRNA and protein results (FIG. 5), the soluble cardiac in vitro GRK activity against the GPCR substrate rhodopsin was significantly reduced in the LV post-LVAD (FIG. 5A). It was previously documented that the soluble GRK activity in cardiac extracts is almost exclusively from βARK1 (Iaccarino et al, Circulation 98:1783-1789 (1998)). The lower βARK1 activity appears to play a role in myocyte recovery after unloading as membrane ISO-stimulated AC activity in these samples was significantly improved (FIG. 5B). Thus, as above in explanted failing human hearts (FIG. 1A), there is an inverse correlation between cardiac GRK activity and βAR signaling and responsiveness.

It is also desirable to determine whether levels of βARK1 found in the blood of LVAD treated patients correlate with cardiac levels and whether lymphocyte βARK1 can be used to monitor functional improvement post-LVAD or help predict myocardial recovery after mechanical unloading. βARK1 has begun to be measured in prepared lymphocytes from LVAD patients. Blood samples and lymphocytes have been obtained from patients prior to LVAD implantation and then again at the time of explantation and cardiac transplantation. Preliminary results in two sets of LVAD patient samples are shown in FIG. 6A. Like cardiac βARK1 protein, lymphocyte levels of βARK1 are reduced substantially by 2 months of LVAD support.

Finally, many studies in animal models of HF have shown that like βARK1, GRK5 is also up-regulated and thus, it may play a role in cardiac signaling and function and be of importance in HF. GRK5 expression levels have been measured in 15 pairs of pre- and post-LVAD cardiac samples and no alterations in GRK5 protein levels after unloading have been found (FIG. 6B). Real-time PCR has also shown no alteration in GRK5 expression levels post-LVAD. These results support the conclusion that βARK1 is the critical GRK involved in regulation of cardiac βAR signaling and function and of importance in HF.

Summarizing, the data above demonstrate that in failing human hearts, LVAD support is associated with decreased levels of βARK1 mRNA, protein, and GRK activity that can be reproduced in the lymphocytes of these patients and provide a possible mechanism for the restoration of βAR signaling and reverse remodeling after mechanical unloading in the failing heart.

All documents and other information sources cited above are hereby incorporated in their entirety by reference.

What is claimed is:

1. A method of monitoring the cardiac β-adrenergic receptor kinase (βARK1) level in a patient comprising monitoring the level of βARK1 in lymphocytes of said patient, wherein an alteration in the lymphocyte level of βARK1 is indicative of an alteration in the cardiac βARK1 level in said patient.

2. The method according to claim 1 wherein the level of βARK1 in said lymphocytes is determined by assaying the βARK1 protein level in said lymphocytes.

3. The method according to claim 2 wherein said βARK1 protein level is assayed by an ELISA.

4. The method according to claim 2 wherein said βARK1 protein level is assayed by Western blotting using βARK1 specific antibodies.

5. The method according to claim 1 wherein the level of βARK1 in said lymphocytes is determined by assaying the βARK1 mRNA level in said lymphocytes.

6. The method according to claim 5 wherein said βARK1 mRNA level is assayed by quantitative RT-PCR.

7. The method according to claim 1 wherein said patient is suffering from heart failure.

8. A method of monitoring cardiac function in a patient suffering from heart failure comprising comparing the level of βARK1 in lymphocytes of said patient at first and second points in time, wherein a reduction in the lymphocyte βARK1 level at said second point in time relative to said first point in time is indicative of an improvement in cardiac function in said patient at said second point in time, and wherein an elevation in the lymphocyte βARK1 level at said second point in time relative to said first point in time is indicative of a diminution in cardiac function in said patient at said second point in time.

9. The method according to claim 8 wherein said first and second points in time are prior to and subsequent to treatment of said patient for heart failure, respectively, wherein a lack of change in, or an elevation of, said lymphocyte βARK1 level at said second point in time relative to said first point in time is indicative of a lack of response to said treatment.

10. A method of monitoring the cardiac level of βARK1 activity in a patient comprising monitoring the level of βARK1 activity in lymphocytes of said patient, wherein an alteration the lymphocyte level of βARK1 activity is indicative of an alteration in the cardiac level of βARK1 activity in said patient.

11. The method according to claim 10 wherein said patient is suffering from heart failure.

12. A method of monitoring cardiac function in a patient suffering from heart failure comprising comparing the level of βARK1 activity in lymphocytes of said patient at first and second points in time, wherein a reduction in the lymphocyte βARK1 activity level at said second point in time relative to said first point in time is indicative of an improvement in cardiac function in said patient at said second point in time, and wherein an elevation in the lymphocyte βARK1 activity level at said second point in time relative to said first point in time is indicative of a diminution in cardiac function in said patient at said second point in time.

13. The method according to claim 12 wherein said first and second points in time are prior to and subsequent to treatment of said patient for heart failure, respectively, wherein a lack of change in, or an elevation of, said lymphocyte βARK1 activity at said second point in time relative to said first point in time is indicative of a lack of response to said treatment.
